(19) United States
(12) Patent Application Publication
(10) Pub. No.: US 2024/0207420 A1
(43) Pub. Date: Jun. 27, 2024

(54) ENZYMATICALLY-CLEAVABLE GLYCIDYL METHACRYLATE HYALURONIC ACID

(71) Applicant: University of Florida Research Foundation, Inc., Gainesville, FL (US)

(72) Inventors: Christine E. Schmidt, Gainesville, FL (US); Mary M. Kasper, Gainesville, FL (US)

(21) Appl. No.: 18/555,956

(22) PCT Filed: Apr. 20, 2022
(86) PCT No.: PCT/US2022/071823

§ 371 Date: Oct. 18, 2023

Related U.S. Application Data
(60) Provisional application No. 63/177,612, filed on Apr. 21, 2021.

(51) Int. Cl. A61K 47/61; A61K 9/06; A61K 38/36; A61K 47/46

(52) U.S. Cl. CPC A61K 47/61; A61K 9/06; A61K 38/363; A61K 47/46

(57) ABSTRACT

Disclosed herein is a hydrogel comprising hyaluronic acid polymers that are at least partially crosslinked with fibrinogen molecules. Also disclosed is a composition comprising a glycidyl methacrylate hyaluronic acid monomer chemically conjugated to a fibrinogen molecule. Also disclosed is a method for producing a hydrogel that involves combining the disclosed composition containing GMHA-Fib precursors with an effective amount of a photoinitiator and light (e.g. UV light) to crosslink unconjugated methacrylate sites through free-radical polymerization. Also disclosed is a method for treating a subject with a peripheral nerve injury that involves implanting the disclosed hydrogel at the site of the injury to promote neural regeneration.

### CROSS-REFERENCE TO RELATED APPLICATIONS

This application claims benefit of U.S. Provisional Application No. 63/177,612, filed Apr. 21, 2021, which is hereby incorporated herein by reference in its entirety.

### BACKGROUND

Peripheral nerve injuries affect approximately 3% of population, which includes traumatic accidents, vascular diseases (e.g. diabetes, peripheral arterial disease), and tumor excision. Clinical repair strategies are limited in their ability for modification (size, integration of growth factors, cells, electrical signals, etc.). Biomaterial strategies offer greater manufacturing control with generally lower processing costs. Glycidyl Methacrylate Hyaluronic Acid (GMHA) can produce a photo crosslinkable hydrogel and combined with an interpenetrating network of collagen I. However, implanted HA hydrogels show insufficient scaffold degradation and reduced cellular infiltration. Improved hyaluronic acid-based hydrogels are needed with enhanced degradation rate.

### SUMMARY

Disclosed herein is a hydrogel comprising hyaluronic acid polymers that are at least partially crosslinked with fibrinogen molecules. This provides two different degradable substrates (HA by hyaluronidase, and fibrinogen by plasmin and MMPs).

In some embodiments, the hyaluronic acid polymer is partially crosslinked with glycidyl methacrylate. In some embodiments, the hydrogel does not comprise collagen, such as collagen I.

Also disclosed is a composition comprising a glycidyl methacrylate hyaluronic acid monomer chemically conjugated to a fibrinogen molecule. In some embodiments, the fibrinogen molecule is conjugated to the glycidyl methacrylate hyaluronic acid monomer by a click-chemistry reaction.

Also disclosed is a method for producing a hydrogel that involves combining the disclosed composition containing GMHA-Fib precursors with an effective amount of a photoinitiator and light (e.g. UV light) to crosslink unconjugated methacrylate sites through free-radical polymerization.

Examples of suitable photoinitiators used with HA include 1-[4-(2-hydroxyethoxy)-phenyl]-2-hydroxy-2-methyl-1-propanone (Irgacure 2959) and lithium phenyl-2,4,6-triethylbenzoylphosphinate (LAP).

Also disclosed is a method for treating a subject with a peripheral nerve injury that involves implanting the disclosed hydrogel at the site of the injury to promote neural regeneration. In some embodiments, the peripheral nerve injury involves a traumatic accident, a vascular disease (e.g. diabetes, peripheral arterial disease), or tumor excision. In some embodiments, the hydrogel is wrapped in a nerve guidance conduit prior to implantation. Biomaterials that can serve as a nerve guidance conduit are known in the art. For example, the nerve guidance conduit can be decellularized porcine small intestine submucosa.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows nerve cross-section of implanted Glycidyl Methacrylate Hyaluronic Acid (GMHA) Collagen (GMHA Col) hydrogel at 6 weeks.

FIG. 2 contains schematics comparing GMHA to GMHA-Fibrinogen.

FIGS. 3A and 3B show the Michael addition reaction of fibrinogen conjugated to glycidyl methacrylate hyaluronic acid (FIG. 3A), and formation of GMHA and GMHA-Fib hydrogels in the presence of a photoinitiator and UV light (FIG. 3B).

FIGS. 4A to 4C show radius of gyration (FIG. 4A), hydrodynamic radius (FIG. 4B), and diffusion coefficient (FIG. 4C) for fibrinogen, GMHA, and GMHA-fibrinogen.

FIGS. 5A and 5B show steady-state moduli (FIG. 5A) and storage moduli as a function of angular frequency (FIG. 5B) of GMHA and GMHA-fibrinogen

FIG. 6 show the full reaction diagram of GMHA-Fibrinogen conjugation.

FIGS. 7A to 7D show the hydrodynamic radius (FIG. 7A), diffusion coefficient of GMHA, fibrinogen, and GMHA-Fib (FIG. 7B), GMHA-Fib hydrogels after UV crosslinking and thrombin gelation (FIG. 7C), and turbidity measurements of GMHA-Fib in thrombin when crosslinked and uncrosslinked with UV light (FIG. 7D).

FIGS. 8A to 8F show the physical characterization of GMHA-Fib hydrogels, including scanning electron microscopy micrographs at 400× of 20GMHA-Fib (FIG. 8A), 40GMHA-Fib (FIG. 8B), 60GMHA-Fib (FIG. 8C), and 20GMHA (FIG. 8D) hydrogel surfaces. Effective crosslinking density (FIG. 8E) and mesh size (FIG. 8F) of varying concentrations of GMHA-Fib compared to GMHA only.

FIGS. 9A to 9F show the mechanical characterization of GMHA-Fib hydrogels compared to GMHA only hydrogels. Rheological frequency sweep from 0.1-10 rad/s of GMHA-Fib and GMHA hydrogels at all frequencies (FIG. 9A) and between GMHA-fib groups at 0.1 rad/s (FIG. 9B), 1 rad/s (FIG. 9C) and 10 rad/s (FIG. 9D). Instantaneous modulus (FIG. 9E) and steady-state modulus (FIG. 9F) GMHA-Fib and GMHA hydrogels.

FIGS. 10A to 10B show hydrogel breakdown in physiologically relevant enzyme solutions containing 0.1 U/ml hyaluronidase (FIG. 10A), and in 0.1 U/ml hyaluronidase and 0.1 U/ml plasmin (FIG. 10B).

FIGS. 11A to 11E show live/dead analysis of rat Schwann cells grown on top of 60 mg/ml GMHA-Fib (FIG. 11A), GMHA (FIG. 11B), and GMHA-Collagen (GMHA-Col) (FIG. 11C) hydrogels after 14 days, scale bar=100 μm. AlamarBlue normalized absorbance at Day 10 (FIG. 11D) and % area covered at Day 14 (FIG. 11E) of Rat Schwann cells grown on GMHA-Fib, GMHA, and GMHA-Col hydrogels.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### GMHA-Fibrinogen Synthesis Protocol

The following is an example protocol for GMHA-Fibrinogen synthesis.

Remove GMHA from −20° C. freezer and place in desiccator for approximately 10-15 minutes. Dissolve GMHA in 8M urea 1×PBS overnight at 20 mg/mL. The next day, remove fibrinogen from −80° C. freezer and place in desiccator for approximately 10-15 minutes. In order to quantify the amount of fibrinogen, run a BCA assay to determine the protein fraction of the fibrinogen stock solution (should be around 60-80%). Dissolve fibrinogen in 8M urea-PBS. Mass TCEP and then dissolve in 8M urea-PBS. Pipet TCEP solution in fibrinogen solution. Add stir bar and stir slowly for 15 minutes on magnetic stir plate. Adjust pH of fibrinogen solution to pH 8 using the pH probe and 5M NaOH (or whatever other base is available). Gently pipette fibrinogen solution to GMHA solution. Keep covered in foil and stir overnight in 4° C. fridge on stir plate.

The next day, dilute GMHA-fibrinogen solution with 8M urea-PBS (double the volume of solvent) and continue to stir for a couple of minutes until solution appears to be mostly homogenous. As performed in the GMHA synthesis protocol, fill a 1 L beaker with approximately 700-800 mL of acetone and place in the chemical fume hood. Using a glass stir rod, stir acetone while gently pouring GMHA-fibrinogen solution into acetone for precipitation. GMHA-fibrinogen should precipitate around the glass stir rod. Gently scrape off precipitate into a glass dish. Wash with fresh acetone and flatten with metal spatula. Repeat 2-3 times. Mass precipitate and re-dissolve product in 8M urea-PBS at 50 mL urea-PBS/g of product in a clean 100 ml bottle. Add stir bar and place on stir plate in 4° C. fridge for several hours until dissolved (should take around 6-8 hours). Dialyze against 1×PBS over 24 hours (3×change) in 4° C. fridge. The next day, continue with dialysis buffer changes. Cover tubes with foil and lyophilize for approximately 4 days depending on the amount of product. Following lyophilization, cover (with foil) and store in GMHA-fibrinogen −20° C. freezer until use.

### GMHA-Fibrinogen Hydrogel Protocol

The following is an example protocol for GMHA-Fibrinogen hydrogel production.

Remove GMHA-fib from −20° C. freezer and place in desiccator for approximately 10-15 minutes. Prepare 1% stock of Lithium phenyl-2,4,6-trimethylbenzoylphosphinate (LAP) photoinitiator, and cover with foil to protect from light. Foil wrap glass vial, and dissolve GMHA-fib at desired concentration in 0.3% LAP and ddlH₂O. Example. For a 1 ml hydrogel solution with a desired concentration of 20 mg/ml GMHA-fib: dissolve 20 mg GMHA-Fib with 300 μl 1% LAP solution+700 μl ddlH₂O. Pipette dissolved hydrogel solution into a mold. Crosslink hydrogel solution under UV light (365 nm, 18.5-22 mW/cm²) for 10 mins. This mechanism crosslinks any free methacrylate groups, not previously conjugated with fibrinogen molecules. Store in PBS until further use

### Specific Embodiments

Embodiment 1. A hydrogel comprising hyaluronic acid polymers at least partially crosslinked with fibrinogen molecules.

Embodiment 2. The hydrogel of embodiment 1, wherein the hyaluronic acid polymer is partially crosslinked with glycidyl methacrylate.

Embodiment 3. The hydrogel of embodiment 1 or 2, wherein the hydrogel does not comprise collagen.

Embodiment 4. A composition comprising a glycidyl methacrylate hyaluronic acid monomer chemically conjugated to a fibrinogen molecule.

Embodiment 5. The composition of embodiment 4, wherein the fibrinogen molecule is conjugated to the glycidyl methacrylate hyaluronic acid monomer by a click-chemistry reaction.

Embodiment 6. The composition of embodiment 5, further comprising a photoinitiator.

Embodiment 7. The composition of embodiment 6, wherein the photoinitiator lithium phenyl-2,4,6-trimethylbenzoylphosphinate (LAP).

Embodiment 8. A method for producing a hydrogel, comprising combining the composition of embodiment 4 or 5 with an effective amount of a photoinitiator and ultraviolet light to crosslink unconjugated methacrylate sites through free-radical polymerization.

Embodiment 9. A method for treating a subject with peripheral nerve injury, comprising implanting the hydrogel of any one of embodiments 1 to 3 at the site of the injury to promote neural regeneration.

Embodiment 10. The method of embodiment 9, wherein the peripheral nerve injury involves a traumatic accident, a vascular disease, or tumor excision.

Embodiment 11. The method of embodiment 10, wherein the vascular disease is a peripheral arterial disease.

Embodiment 12. The method of embodiment 11, wherein the subject has diabetes.

Embodiment 13. The method of any one of embodiments 9 to 12, wherein the hydrogel is wrapped in a nerve guidance conduit prior to implantation.

Embodiment 14. The method of embodiment 13, wherein the nerve guidance conduit comprises decellularized porcine small intestine submucosa.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

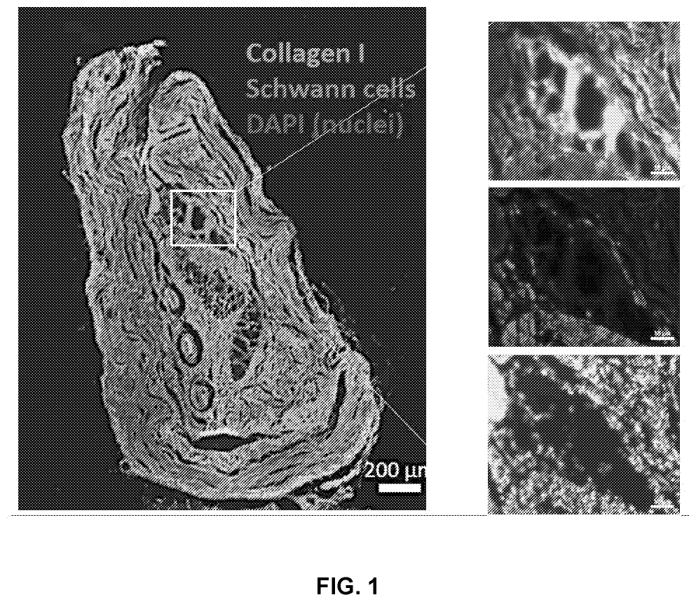

Implanted GMHA-Col hydrogels show insufficient scaffold degradation at 6 weeks post implantation (FIG. 1). This results in little cellular infiltration and residual hydrogel remaining within the implant space.

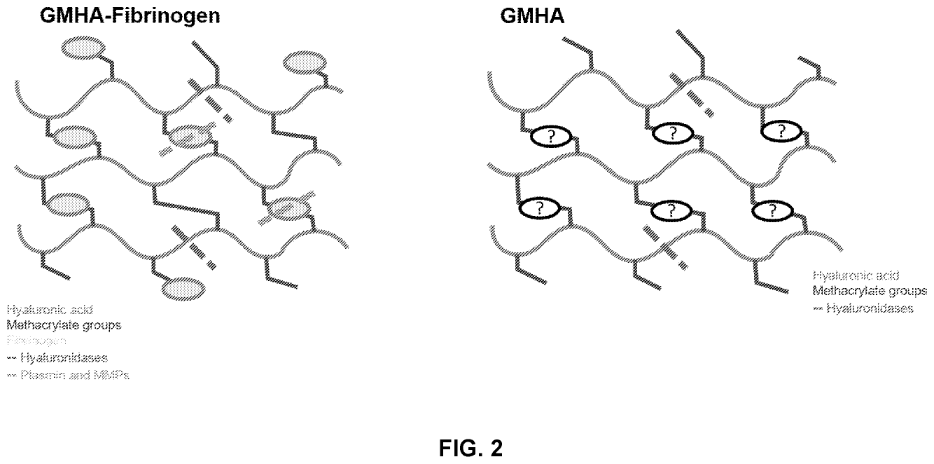

GMHA only has one mode of degradation on HA backbone (by hyaluronidases). Covalent methacrylate links cannot be broken. Conjugation of fibrinogen to GMHA would yield two degradable substrates (FIG. 2). Intrinsically cell adhesive fibrinogen molecule would also eliminate the need for collagen.

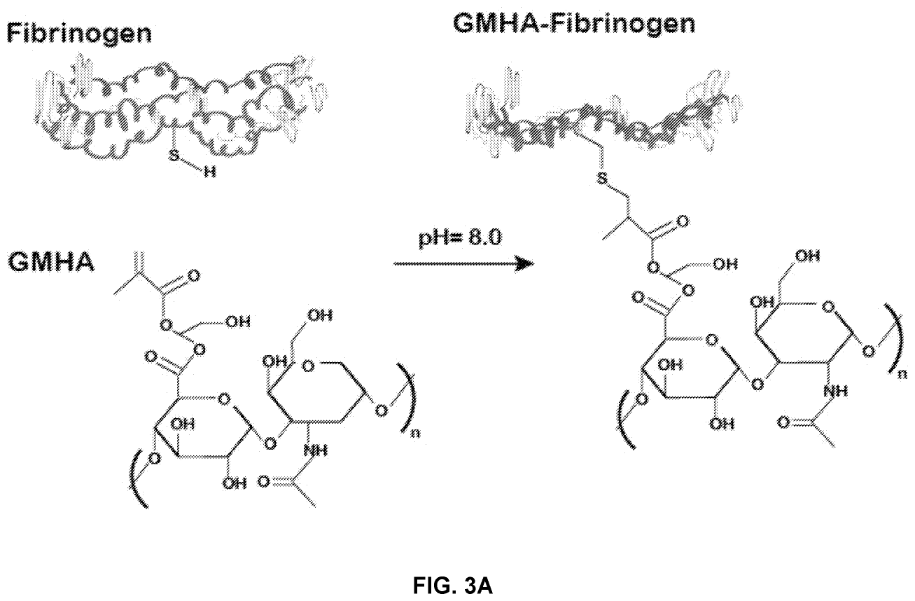

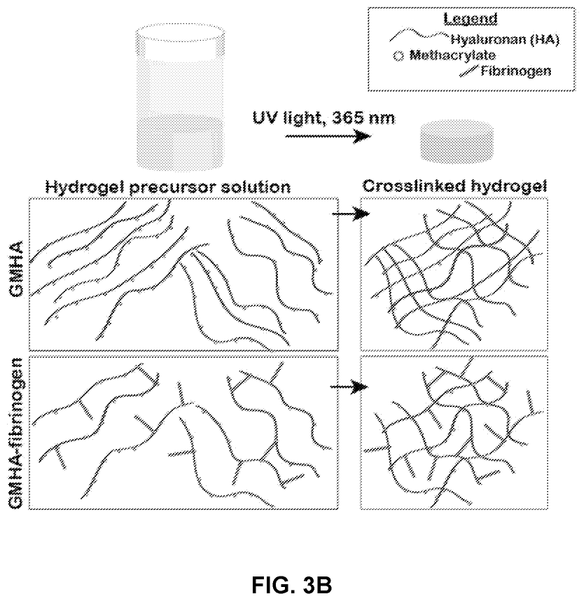

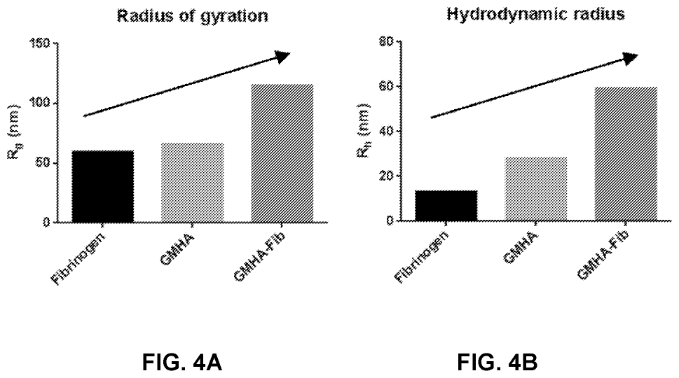

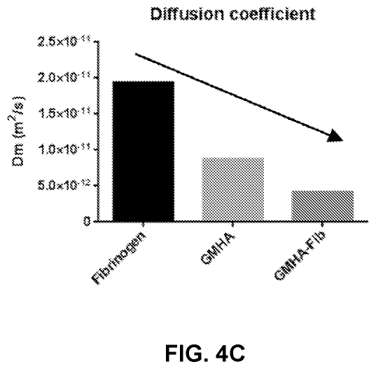

Fibrinogen can be successfully conjugated to modified HA through a click reaction (FIG. 3A). Dynamic light scattering analysis was used to confirm conjugation (FIGS. 4A-4C). There was an increase radius of gyration and hydrodynamic radius (FIGS. 4A-4B), and a decreased diffusion coefficient (FIG. 4C).

The GMHA-Fib precursor was then combined with a photoinitiator (e.g. UV exposure), which resulted in the free, unconjugated methacrylate sites crosslinking through free-radical polymerization (FIG. 3B).

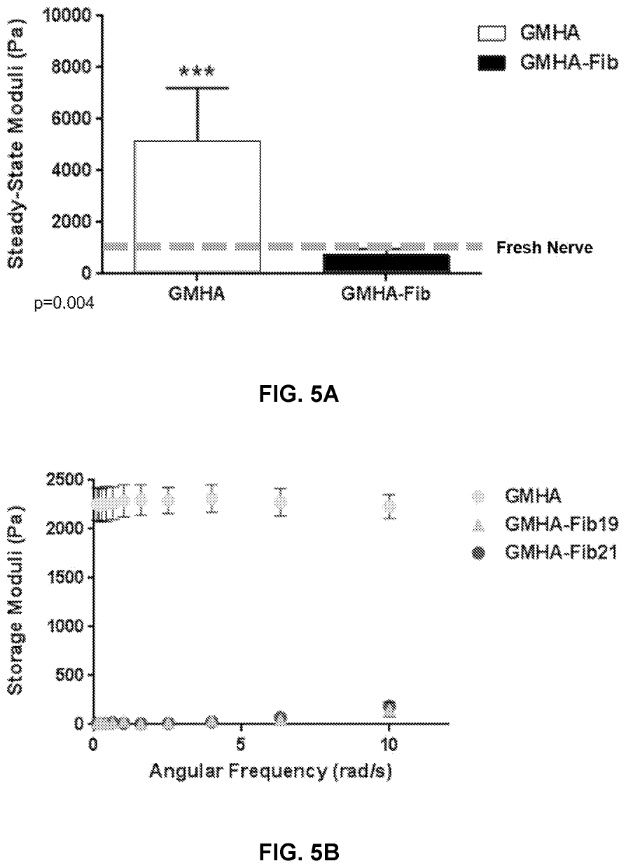

The GMHA-Fib bioconjugate resulted in weaker mechanical properties (FIGS. 5A and 5B). Similar mechanical trends were observed through indentation and rheological testing. GMHA-Fib mechanical properties matched to nerve. Fewer methacrylate crosslinks decreased mechanical properties of GMHA-Fib. No significant differences of rheological properties were observed between GMHA-fib batches.

### Example 2

### Introduction

A significant tissue engineering challenge is tuning the rate of scaffold degradation to match the rate of ECM deposition and remodeling. Many hydrogel scaffolds have been synthesized from natural materials and their derivatives such as collagen, gelatin, HA, and fibrin to incorporate inherent enzymatically active substrates. However, the design of hydrogel scaffolds often involves chemically modifying natural materials to address other criteria (e.g., mechanical strength) and often results in limiting the availability of bioactive sites, including adhesive and degradable linkages (Sahoo, S., et al. Biomacromolecules. 2008 9:1088-1092). Likewise, synthetically derived hydrogels are faced with degradability challenges because they inherently lack biodegradable substrates; thus, most modified scaffolds degrade by hydrolysis of the polymer backbone (Sahoo, S., et al. Biomacromolecules. 2008 9:1088-1092; Meyvis, T. K. L., et al. Macromolecules. 2000 33:4717-4725). A simplistic strategy to improve hydrolytic degradation is to reduce the number of crosslinks within the scaffold, however, this strategy fails to facilitate active, targeted degradation and often compromises other functional properties of the scaffold such as stiffness (Meyvis, T. K. L., et al. Macromolecules. 2000 33:4717-4725; Hutson, C. B., et al. Tissue Eng. 2011 17:1713-1723). To address these challenges, researchers have investigated the incorporation of enzymatically sensitive substrates that can allow for tunable degradation in the body by specific enzymes, proteases, and matrix metalloproteinases (MMPs).

The exploitation of proteolysis has prompted the development of MMP-cleavable scaffolds by conjugating MMP-sensitive peptide sequences to hydrogel matrix backbones (Patterson, J., et al. Biomaterials. 2010 31:7836-7845; Lutolf, M. P., et al. Proc. Natl. Acad. Sci. U.S.A 2003 100:5413-5418; Seliktar, D., et al. J. Biomed. Mater. Res. 2004 68:704-716; Feng, Q., et al. PLOS One. 2014 9; Chau, Y., et al. Biomaterials. 2008 29:1713-1719; Aimetti, A. A., et al. Biomaterials. 2009 30:6048-6054; Mann, B. K., et al. Biomaterials. 2001 22:3045-3051). Hubbell pioneered the use of polyethylene glycol-vinyl sulfone (PEG-VS) modified MMP-sensitive conjugates by Michael addition reaction (Patterson, J., et al. Biomaterials. 2010 31:7836-7845; Seliktar, D., et al. J. Biomed. Mater. Res. 2004 68:704-716). The degradation of PEG, a synthetic polymer, was rendered tunable with the addition of MMP-cleavable substrates. Patterson and Hubbell (2010) demonstrated that by changing the cleavable peptide sequence, a hydrogel could be rendered relatively degradable with tunable specificity to different MMP species. Furthermore, hydrogels with faster, targeted degradation resulted in better cell spreading and increased proliferation in vitro (Patterson, J., et al. Biomaterials. 2010 31:7836-7845; Seliktar, D., et al. J. Biomed. Mater. Res. 2004 68:704-716). The extensive work in MMP-cleavable chemistries has motivated the fabrication of other enzymatically-cleavable chemistries including plasmin (West, J. L., et al. Macromolecules. 1999 32:241-244; Dikovsky, D., et al. Biomaterials. 2006 27:1496-1506; Peled, E., et al. J. Biomed. Mater. Res. 2006 80A:874-884; Almany, L. et al. Biomaterials. 2005 26:2467-2477) and chymotrypsin-sensitive peptide conjugates (Lin, C. C., et al. Biomaterials. 2011 32:9685-9695).

Although enzymatically cleavable peptide sequences are advantageous for providing targeted degradation to a scaffold matrix, the incorporation of other biologically functional properties, such as cell adhesion, requires the conjugation of other peptide sequences (e.g., RGD). Thus, it may be advantageous to incorporate whole proteins that possess multiple functional properties of interest. The successful fabrication of PEG hydrogels with fibrinogen conjugates (PEG-fibrinogen) to exploit the natural wound healing mechanisms of fibrin has been demonstrated (Dikovsky, D., et al. Biomaterials. 2006 27:1496-1506; Peled, E., et al. J. Biomed. Mater. Res. 2006 80A:874-884; Almany, L. et al. Biomaterials. 2005 26:2467-2477; Pradhan, S., et al. J. Biomed. Mater. Res. Part A. 2016 105). PEG-fibrinogen hydrogels showed varying degrees of proteolytic resistance based on relative amounts of PEG to fibrinogen (Dikovsky, D., et al. Biomaterials. 2006 27:1496-1506). Additionally, five weeks following hydrogel implantation into a tibial defect in rats, histological analysis showed good osteogenesis and complete scaffold degradation (Peled, E., et al. J. Biomed. Mater. Res. 2006 80A:874-884).

PEG and other synthetic materials have been used extensively in tissue engineering; however, it may be beneficial to incorporate a natural-based material as the polymer matrix backbone to augment the scaffold's functional properties through its inherently bioactive composition (Reddy, M. S. B., et al. Polymers 2021 13). In addition, upon implantation, a material that mimics the native ECM can further stimulate endogenous wound healing cascades that are conducive for regeneration. Natural materials, including HA (Feng, Q., et al. PLOS One. 2014 9; Park, J., et al. J. Biomed. Mater. Res. 2010 93:1091-1099) and self-assembling peptides (Chau, Y., et al. Biomaterials. 2008 29:1713-1719), have been modified with MMP-cleavable conjugates to better improve degradation. HA conjugated with MMP-sensitive sites demonstrated improved matrix remodeling deposition of glycosaminoglycans and collagen in vitro (Feng, Q., et al. PLOS One. 2014 9). A study by Park et al. (2010) showed successful conjugation of MMP-sensitive peptides and laminin-binding peptides to HA hydrogels. Laminin-binding peptides promoted cellular infiltration, where MMPs subsequently degraded the hydrogel to further release encapsulated brain-derived neurotrophic factor (BDNF). In a spinal cord injury model, the HA-IKVAV-MMP+BDNF hydrogel significantly improved locomotor function two weeks following injury, demonstrating further application for drug and molecular delivery (Park, J., et al. J. Biomed. Mater. Res. 2010 93:1091-1099). The utility of HA-conjugated scaffolds is promising because it is easy to incorporate other biological components; however, previous studies rely on the incorporation of multiple peptides with individual roles, as opposed to a singular molecule with multiple functions. The former approach often requires the purchase of extremely costly peptide sequences or require time consuming multi-step syntheses of each individual component to produce the desired peptide sequences in-house.

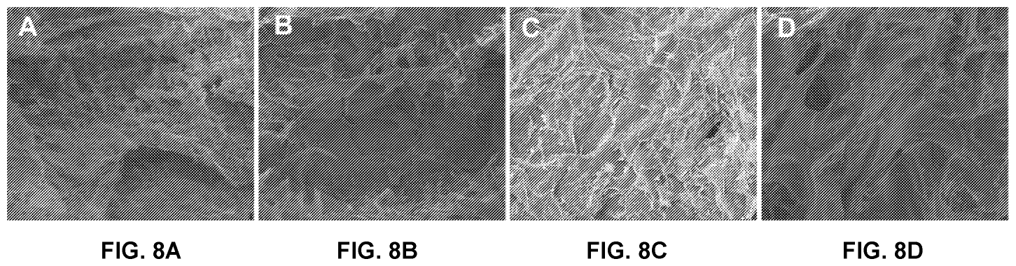

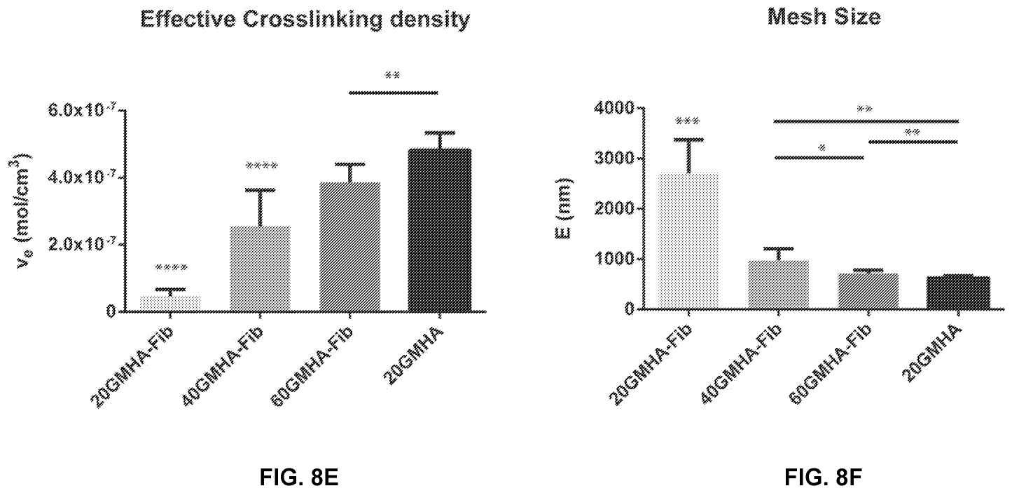

In this work, GMHA chemistry was modified to include a fibrinogen linker (GMHA-Fibrinogen) that can be used to both enhance degradation and provide cell adhesivity. The previous hydrogel formulation used in a peripheral nerve therapeutic (Kasper, M., et al. Biomaterials. 2021 279:121212), GMHA-Col, consisted of a GMHA network with an interpenetrating network of collagen I (not covalently conjugated) to provide cell adhesivity. This previous formulation was unable to address concerns over hydrogel degradation because both matrices were formed independent of one another, and thus needed to be degraded independently of one another. Fibrinogen has been shown to have a natural and reversible binding affinity to HA, making it an ideal linker for GMHA (LeBoeuf, R. D., et al. J. Biol. Chem. 1986 261:12586-12592). Additionally, HA helps to stabilize the provisional fibrin matrix during wound healing (Wang, K. K., et al. Microsurgery. 1998 18:270-275). Fibrin cables are critical to successful regeneration of peripheral nerve, specifically to provide a microarchitectural substrate for infiltrating Schwann cells (Williams, L. R., et al. J. Comp. Neurol. 1983 218:460-470). HA and fibrinogen's strong colocalization would be potentially advantageous for stimulating endogenous regeneration pathways after implantation. The new GMHA-Fibrinogen chemistry is capable of UV crosslinking (FIGS. 8A-8B) to provide mechanical tunability and the ability to incorporate microarchitectural cues through magnetic templating. Whole fibrinogen protein maintains its functional properties after conjugation including fibrin crosslinking, degradation, and cell binding. These findings establish GMHA-Fibrinogen's utility in tissue engineered applications and more specifically, in future peripheral nerve applications.

### Materials and Methods

### GMHA-Fibrinogen Synthesis

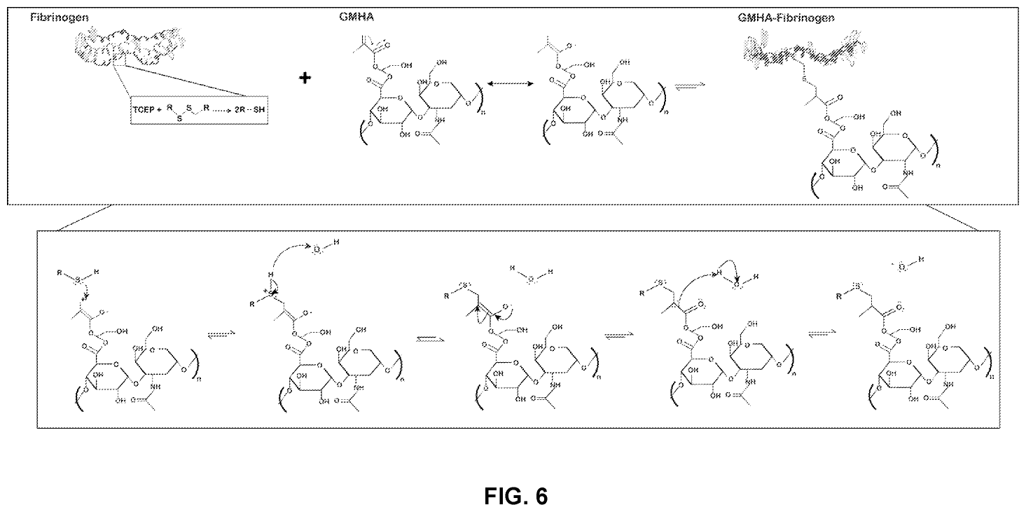

Glycidyl methacrylate hyaluronic acid (GMHA) was synthesized and stored as previously described (Leach, J. B., et al. Biotechnol. Bioeng. 2003 82:578-589). GMHA was dissolved in 8M urea-PBS at 20 mg/ml overnight. A solution of fibrinogen was dissolved at 7 mg/ml with 0.53 mg/ml tris(2-carboxyethyl) phosphine in 8M urea-PBS and slowly mixed on a magnetic stir plate for 15 min. The fibrinogen solution was adjusted to pH 8.0 using 1M NaOH and then added to the GMHA solution and reacted overnight at 4° C. on a magnetic stir plate (FIG. 6). The resulting GMHA-Fibrinogen (GMHA-Fib) solution was further diluted to half its concentration with 8M urea-PBS and stirred for an additional 15 min at room temperature. GMHA-Fib was precipitated in acetone and redissolved in 8M urea-PBS at 30 mg/ml overnight at 4ºC. The redissolved GMHA-Fib solution was dialyzed in PBS for 24 h at 4° C. then lyophilized for 5 days and stored at −20° C. until use.

### Molecular Characterization Via Dynamic Light Scattering Analysis

Dynamic light scattering analysis was performed to determine the hydrodynamic radius and mutual diffusion coefficient. Briefly, samples were dissolved at 0.10 w/v % in 1×PBS and filtered through a 0.45 μm PVDF filter (Millipore) directly into pre-cleaned scattering cells prior to measurement.

Multi-angle dynamic light scattering (DLS) measurements were performed on an ALV/CGS-3 four-angle, compact goniometer system, containing a 22 mW HeNe linear polarized laser operating at a wavelength of λ=632.8 nm and scattering angles from θ=30-150°. Fluctuations in the scattering intensity were measured via an ALV/LSE-5004 multiple tau digital correlator and analyzed via the intensity autocorrelation function (g(2)(T)). The data was fit using a cumulant analysis, and the mutual diffusion coefficient was determined:

$$Γ = {q}^{2} {D}_{m} \qquad ( 4 - 1 )$$

where Γ is the average decay rate of the autocorrelation function and q² is the scalar magnitude of the scattering vector. The hydrodynamic radius (Rₕ) was calculated through the Stokes-Einstein equation

$${D}_{m} \approx {D}_{t} = \frac{{k}_{B} T}{6 {πη}_{s} {R}_{h}} \qquad ( 4 - 2 )$$

where Dₘ is the mutual diffusion coefficient, Dt is the tracer diffusion coefficient, kg is the Boltzmann constant, T is the absolute temperature, and ηₛ is the solvent viscosity (Keller, C. B., et al. Polym. Chem. 2021 12:4758-4769).

GMHA-Fibrinogen Hydrogel Fabrication

GMHA-Fib hydrogels were fabricated by dissolving the desired polymer concentration (20, 40, or 60 mg/ml) in 1×PBS with 0.3% w/v lithium phenyl-2,4,6-trimethylbenzoylphosphinate (LAP) photoinitiator. Hydrogels were injected into 8×1.7 mm silicone molds [Grace Bio-labs, 664201] and placed under a 365 nm UV light with 18-22 mW/cm² intensity for 10 min and stored in 1×PBS until use. Control hydrogels were fabricated with 20 mg/ml GMHA, 0.3% w/v LAP with identical methods described above.

To determine the time for effective fibrin crosslinking, UV-crosslinked GMHA-Fib hydrogels were placed in a crosslinking solution containing 25 U/ml thrombin and 40 mM CaCl₂). Uncrosslinked GMHA-Fib hydrogel precursor solution was directly mixed with the fibrin crosslinking solution to determine fibrin stabilization without the assistance of a preformed GMHA matrix. Absorbance was measured using a SpectraMax M5e Multi-Mode Microplate Reader spectrophotometer [Molecular Devices, USA]. Kinetic measurements were taken every 30 s for 2 h at 450 nm (n=4).

### Scanning Electron Microscopy (SEM)

To prepare hydrogels for SEM analysis, samples were fabricated, immediately flash frozen in liquid nitrogen, and lyophilized. Dried hydrogel samples were mounted on SEM stubs with carbon tape and sputter coated with gold-palladium. Samples were imaged at 5 kV with a FEI Nova NanoSEM 430 at 800×.

### Physical Characterization Via Swelling Analysis

Swelling analysis of hydrogels was performed by taking the weights of the samples immediately after crosslinking, after swelling in water at 25° C. overnight, and after lyophilization to obtain the polymer dry weight (n=8). Flory-Rehner equations were used as previously described by Leach et al. to determine physical properties of hydrogels including effective crosslinking density and mesh size (Leach, J. B., et al. Biotechnol. Bioeng. 2003 82:578-589). Degree of mass swelling (QM) was calculated as the ratio of the mass of hydrogels swollen in water over the dry polymer mass.

$${Q}_{M} = \frac{{M}_{S}}{{M}_{D}} \qquad ( 4 - 3 )$$

Degree of volumetric swelling, Q_{V}. was determined from the degree of mass swelling, the dry polymer density (Pp, estimated at 1.229 g/cm³ for HA), and the solvent density (ρₛ. 1 g/cm³ for water), as calculated in equation 4-4.

$${Q}_{V} = 1 + \frac{{ρ}_{p}}{{ρ}_{s}} ( {Q}_{M} - 1 ) \qquad ( 4 - 4 )$$

Average molecular weight between crosslinks M̅_{c} was approximated from the degree of volumetric swelling using equation 4-5, where υ̅ is the specific volume of the polymer (0.814 for HA), 11 is the molar volume of water (18.1 cm³/mol), and x is the Flory-interaction parameter (approximately 0.473 for HA in water).

$${Q}_{V}^{5 / 3} \approx \frac{\overline{v} {\overline{M}}_{c}}{{v}_{1}} ( \frac{1}{2} - χ ) \qquad ( 4 - 5 )$$

The effective crosslink density (mol/cm³), vₑ, was calculated from the dry polymer density and average molecular weight between crosslinks, as per equation 4-6

$${v}_{e} = \frac{{ρ}_{p}}{{M}_{c}} \qquad ( 4 - 6 )$$

Finally, the mesh size (ξ) was calculated using equation 4-7,

$$ξ \approx {Q}_{V}^{1 / 3} \sqrt{\overline{{r}_{o}^{2}}} \qquad ( 4 - 7 )$$

where r̅ₒ̅²̅ is the root mean square end-to-end distance of HA (Cleland, R. L., et al. Biopolymers. 1970 9:799-810). Thus, the root mean square can be further reduced using equation 4-8, where n is the number of disaccharide repeat units, which is approximated at 5305, using HA with 2 MDa molecular weight.

$$\overline{{r}_{o}^{2}} \approx {( 2.4 nm )}^{2} * 2 n \qquad ( 4 - 8 )$$

Mesh size can then be simplified from equation 4-6 and expressed in nanometers using equation 4-9.

$$ξ \approx 0.1748 \sqrt{{\overline{M}}_{c}} {Q}_{V}^{1 / 3} \qquad ( 4 - 9 )$$

Overall, mesh size calculations and effective crosslink density require approximations, and assumptions such as a homogenous hydrogel network. Therefore, these values are provided as approximations for relative changes in physical properties with given hydrogel compositions.

### Mechanical Characterization of GMHA-Fibrinogen Hydrogels

Stress-relaxation measurements were obtained via bulk indentation testing using a Bruker BioSoft In Situ Indenter. Tests were performed with a 3-mm-diameter spherical glass tip. Hydrogels were tested without submersion but kept hydrated with 1×PBS. Tests were performed by indenting approximately 6.5% of the total sample height at a rate of 20 μm/s. The probe was held at the maximum indent depth for 60 s to obtain stress relaxation data. Three locations were tested per sample, with each experimental group containing six samples (n=6). The Hertz contact model was used to fit the relaxation data to the standard linear solid model to obtain the rate-dependent instantaneous modulus and the steady-state modulus (Stewart, D. C., et al. PLOS One. 2017 12:1-19).

Rheological measurements were obtained using an MCR-302 rheometer (Anton Paar, Graz, Austria). An 8 mm-diameter parallel top plate was lowered onto the hydrogels and any excess hydrogel was trimmed using a spatula. Amplitude sweeps from 0.01-100% strain was performed to determine the linear viscoelastic region. Frequency sweeps from 0.1-100 rad/s and 0.02% strain were performed to determine the storage modulus (n=6-11).

### Hydrogel Proteolytic Degradation

Degradation of hydrogels was determined by obtaining the weights of samples over 90 days. Hydrogels with 20, 40 and 60 mg/ml GMHA-Fib were compared to 20 mg/ml GMHA. Hydrogels were fabricated (n=8) and swollen in 1×PBS overnight at 37° C. on a shaker plate (60 rpm). The weight of hydrogels swollen in PBS were recorded before initiating the assay and used for day 0 measurements. Samples were transferred to a solution containing either 0.1 U hyaluronidase only or 0.1 U hyaluronidase+0.1 U plasmin in 1×PBS and placed on a shaker plate (60 rpm) at 37° C. for the duration of the 90 days. Hydrogel weights were measured daily for the first three days, and then every three days afterward. Enzyme solutions were replenished every three days. The percentage of mass degradation was determined as a ratio of the experimental weight at each time point to the initial weight.

### In Vitro Biocompatibility of GMHA-Fibrinogen Hydrogels

Dry GMHA-Fib polymer was sterilized by supercritical CO₂ with methods adapted from Casali et al. (Casali, D. M., et al. J. Supercrit. Fluids. 2018 131:72-81). Briefly, GMHA-Fib was loaded into the treatment chamber of the supercritical CO₂ apparatus. Liquid carbon dioxide was then compressed in a chilled syringe pump. The valve to the treatment chamber was opened and dry supercritical CO₂ was slowly bubbled into the chamber. During treatment, the environmental chamber was used to maintain the temperature at 40° C., and a back-pressure regulator was used to keep the CO₂ pressure in the vessel constant at 27.6 MPa (4000 psi). After the desired exposure time, the manual hand pump was used to depressurize the treatment chamber to atmospheric pressure and the sterilized polymer was retrieved.

GMHA-Fib and GMHA control hydrogels were fabricated as previously described under sterile conditions and placed in a 48 well plate. GMHA-Collagen (GMHA-Col) hydrogels were also fabricated as a positive control with 20 mg/ml GMHA, 3 mg/ml collagen |[Corning, 354249] and 0.3% w/v LAP. Passage 3 rat Schwann cells (RSCs) were seeded on top of randomized hydrogel samples at 19,000 cells/cm² (n=6) in RSC media. At 14 days, a live/dead assay was performed on cell laden hydrogels. The live/dead solution was prepared with 4 μM ethidium homodimer-1 and 2 μM calcein AM. Samples were rinsed with 1×PBS and then incubated with the live/dead solution at room temperature for 45 min before removing the solution and rinsing with 1×PBS. Cells were imaged at 10× magnification on a Zeiss LSM 880 laser-scanning confocal microscope.

Live/dead image analysis was performed on ImageJ software. ImageJ was used to separate the live and dead channels of the raw images and convert the images to greyscale. Images were thresholded into binary maps; lower and upper thresholds were determined to exclude small artifacts and include most of the cells without over-exposing the image. The “Analyze Particles” feature was used to count total number of cells, measure the total covered area, and percent area covered. Images of both live and dead channels were analyzed to calculate the percent viability.

### Statistical Analysis

All statistical analyses were performed using JMP Pro 14. Shapiro-Wilk and Levene tests were used to test the normality and variance of the experimental group distributions, respectively. Swelling data (i.e., effective crosslinking density and mesh size) had non-normal distributions and non-equal variance and thus did not meet the criteria for a 1-way ANOVA, therefore a Kruskal-Wallis test was used to determine statistically significant differences between the experimental group medians. Pairwise comparisons were made post-hoc with a single-step p-value adjustment, where differences were deemed statistically significant at a confidence level α=0.05. Due to the non-normality of the indentation data, the indentation data was log transformed and analyzed for significance using a 1-way ANOVA. Statistical differences between experimental groups were determined with 1-way ANOVA for rheological and in vitro studies. Analyses were followed by Tukey's Honestly Significant Difference posthoc test for multiple comparisons, with an overall confidence interval α=0.05. All data are represented as the mean±standard deviation (SD).

### Results

### Determination of GMHA-Fib Conjugation Via Dynamic Light Scattering

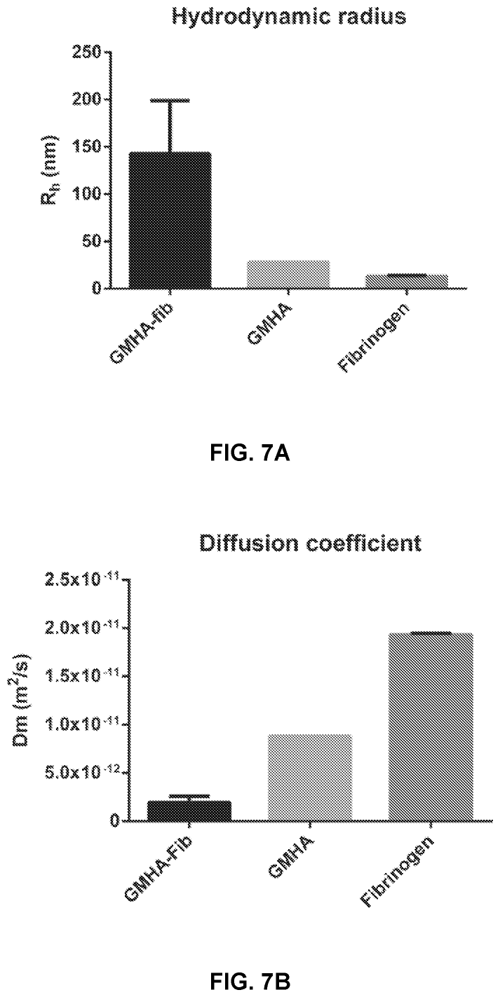

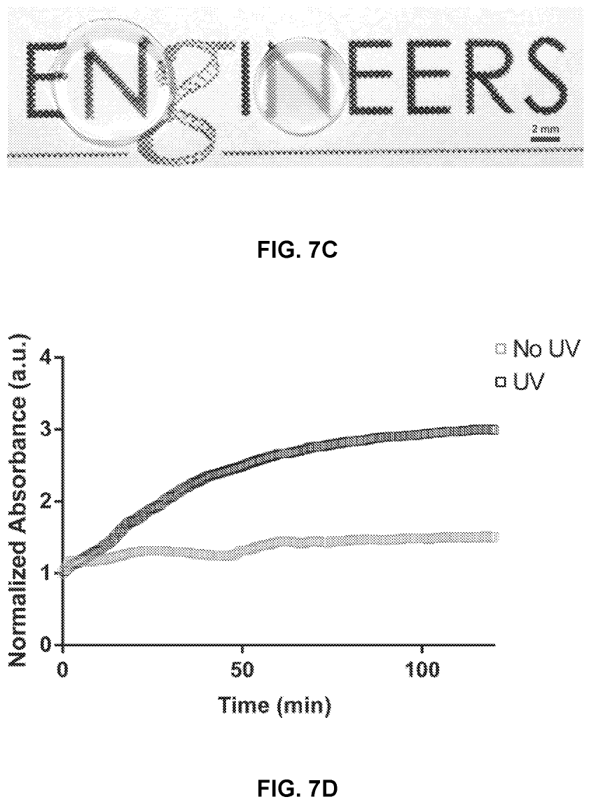

Dynamic light scattering analysis was performed to determine effective conjugation of GMHA-Fib. The hydrodynamic radius of GMHA and fibrinogen were determined to be 28 nm and 13 nm, respectively (FIG. 7A). GMHA-Fib hydrodynamic radius was determined to be 142±56 nm, significantly larger than GMHA or fibrinogen alone. Accordingly, the diffusion coefficient of GMHA and fibrinogen were determined to be 8.81×10⁻¹² and 8.81×10⁻¹¹, respectively (FIG. 7B). The GMHA-Fib diffusion coefficient was determined to be 1.93×10-12±6.78×10⁻¹³, which is significantly lower than coefficients for pure GMHA and fibrinogen components.

### Hydrogel Formation and Fibrin Matrix Stabilization

Fibrin activity was confirmed by placing hydrogels in a crosslinking solution containing thrombin (FIG. 7C). When placed in thrombin, hydrogels were observed to geometrically shrink in diameter. Additionally, hydrogels became opaque, a characteristic of fibrin crosslinked hydrogels (Potier, E., et al. J. Mater. Sci. 2010 45:2494-2503; Suenson, E., et al. Eur. J. Biochem. 1984 140:513-522). Turbidity measurements were obtained by measuring the change in absorbance of GMHA-Fib UV-crosslinked hydrogels or precursor solution placed in thrombin crosslinking solution (FIG. 7D). UV-formed hydrogels exhibited a considerable increase in absorbance, however the precursor solution mixed with thrombin crosslinking solution did not show changes in absorbance over time.

### Structural Analysis of GMHA-Fib Hydrogels

Scanning electron microscopy was used to visualize various hydrogel compositions. A more fibrous morphology can be observed in 40GMHA-Fib (FIG. 8B) and 60GMHA-Fib (FIG. 8C) compared to 20GMHA-Fib (FIG. 8A) and 20GMHA (FIG. 8D). 20GMHA-Fib contains many pores compared to 20GMHA that has a smoother surface appearance.

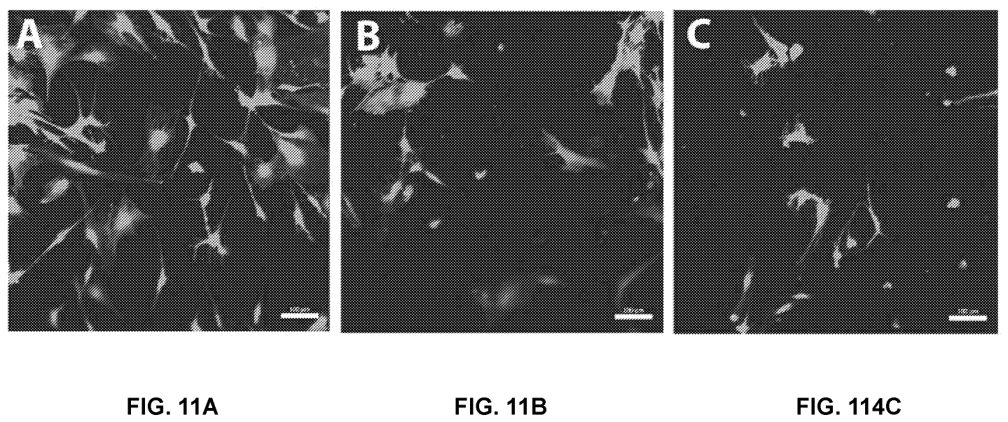

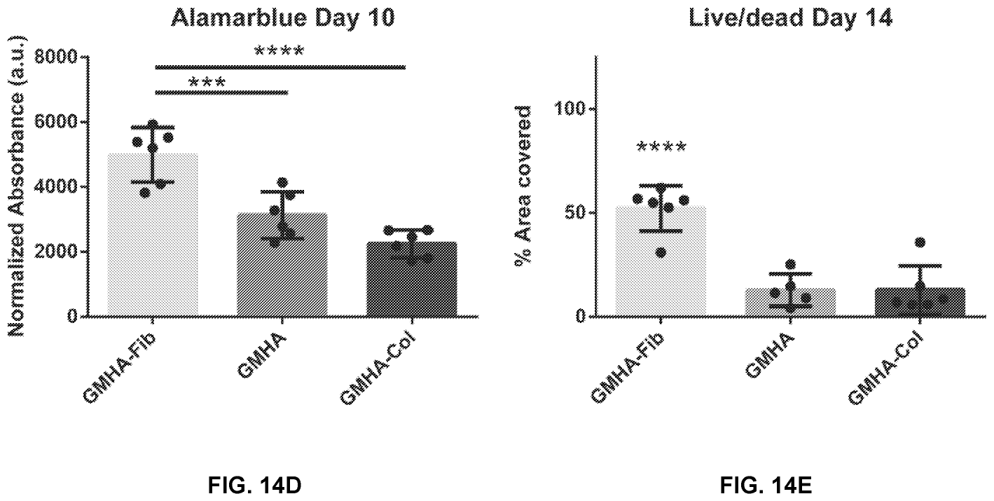

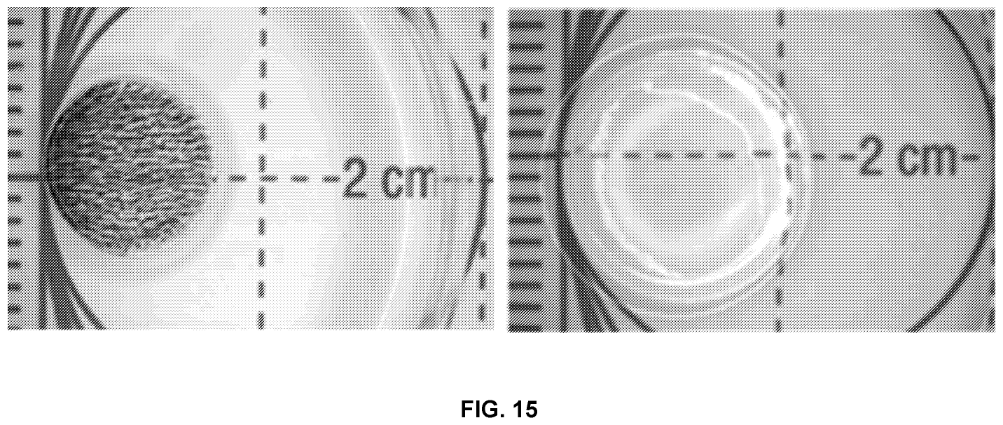

Flory-Rehner swelling analyses were performed to determine the effective crosslinking density (FIG. 11E) and average mesh size (FIG. 11F) of GMHA-Fib hydrogels compared to GMHA only. The mesh size is defined as the distance between molecules, whereas effective crosslinking density is defined as the number of crosslinks per volume. By increasing the polymer concentration of GMHA-Fib, the effective crosslinking density significantly increases; however, even at the highest GMHA-Fib concentration, hydrogels have a significantly lower crosslinking density than 20GMHA hydrogels. Mesh size is inversely related to crosslinking density and appears to decrease with increasing GMHA-Fib concentration, although there appear to be no differences in the mesh size between 40GMHA-Fib and 60GMHA-Fib. All GMHA-Fib groups have a larger mesh size than GMHA only.

### Mechanical Evaluation of GMHA-Fib Hydrogels

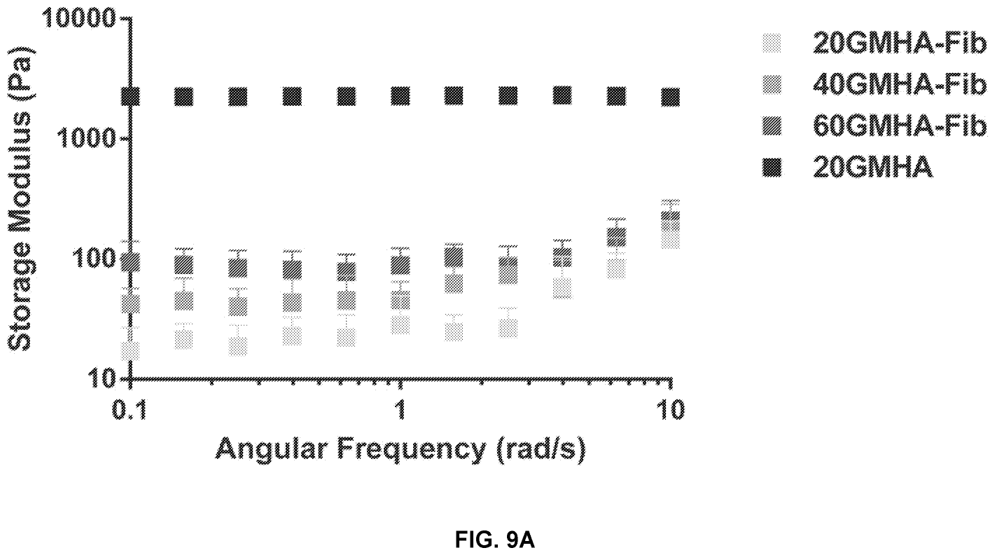

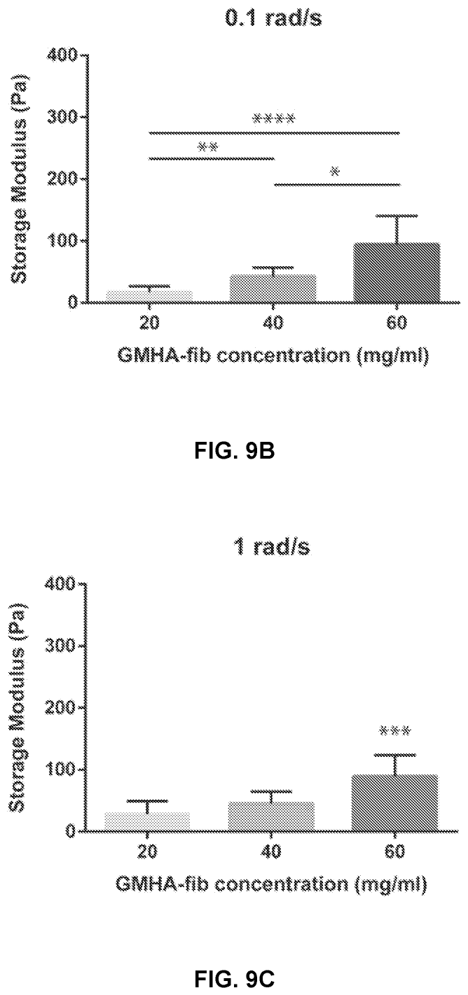

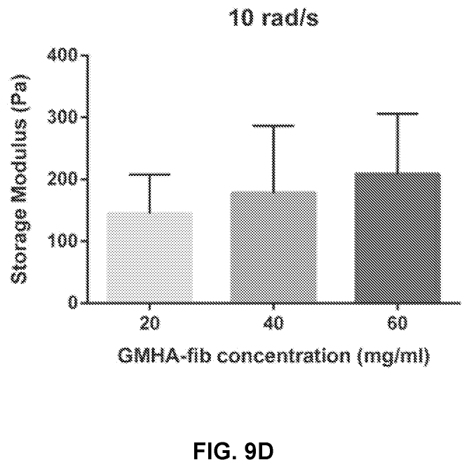

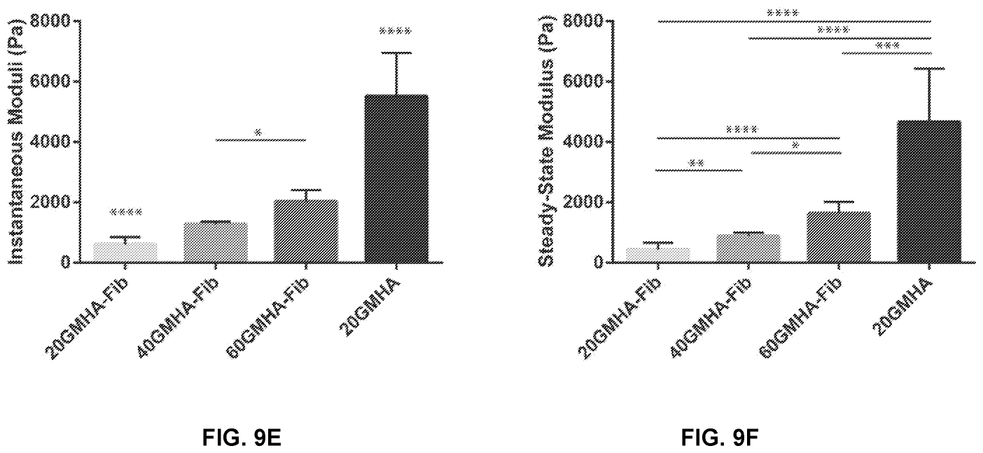

Mechanical properties of GMHA-Fib hydrogels were measured using traditional rheological testing and bulk non-destructive indentation testing. Rheological measurements of the storage modulus from 0.1-10 rad/s show GMHA-Fib hydrogels are significantly less stiff than GMHA only hydrogels (FIG. 12A). There appear to be no significant differences in the storage modulus between GMHA-Fib groups at high frequencies (FIG. 12D), while greater differences are observed between groups at lower frequencies (FIGS. 9B, 9C). The mechanical stiffness was significantly different between all groups when measured with indentation (FIGS. 9E, 9F). Data obtained through indentation corroborates with rheological data that also illustrate that the GMHA hydrogel steady-state modulus is significantly stiffer than GMHA-Fib conjugates. Moreover, there is an overall decrease between the instantaneous to steady-state modulus.

### Hydrogel Degradation Mediated by Fibrinogen Linker

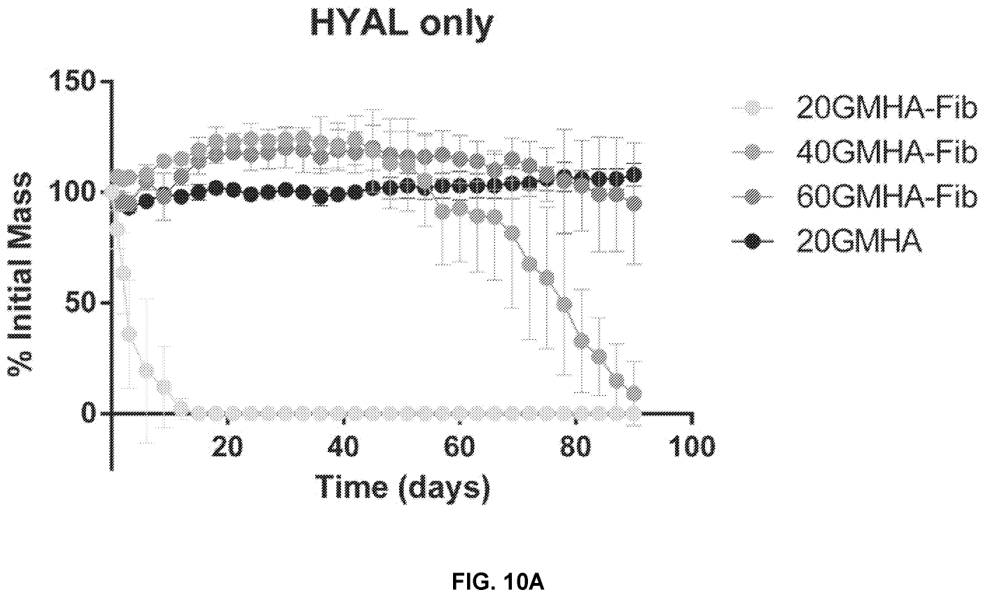

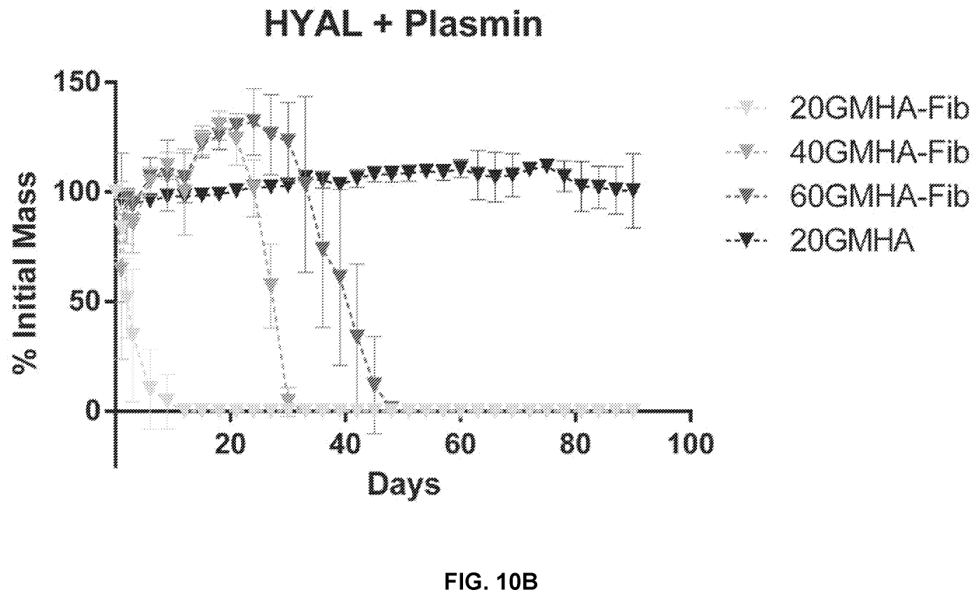

Mass measurements of GMHA-Fib and GMHA hydrogels were taken to evaluate hydrogel degradation over ninety days. Hydrogels were either placed in an enzyme solution containing hyaluronidase only (FIG. 10A) or a combination of hyaluronidase and plasmin (FIG. 10B) to target the hyaluronan and fibrinogen (Suenson, E., et al. Eur. J. Biochem. 1984 140:513-522) matrices, respectively. GMHA hydrogels placed in either solution maintained ˜100% of their weight through the 90-day assessment period. 20GMHA-Fib hydrogels degraded readily in solutions containing either hyaluronidase only (complete degradation by day 12) or hyaluronidase and plasmin (degradation by day 9), likely due to its low crosslinking density and physical handling required for measurements. Except for 20GMHA-Fib hydrogels, all GMHA-Fib hydrogels in either solution exhibited a moderate increase in their weight by ˜20-30% of their initial weight before characteristic loss in mass attributed to degradation. 40GMHA-Fib in hyaluronidase degraded slowly, yet consistently, with approximately 9% of its weight remaining by 90 days, whereas 60GMHA-Fib still maintained approximately 95% of its initial weight. When placed in a solution containing both hyaluronidase and plasmin, all GMHA-Fib hydrogels degraded with 20GMHA-Fib degrading completely by day 12, 40GMHA-Fib degrading completely by day 33, and 60GMHA-Fib degrading completely by day 51.

### Biocompatibility of GMHA-Fibrinogen In Vitro

Representative micrographs of RSCs grown on hyaluronan based hydrogels illustrate poorer cell growth on GMHA (FIG. 11B) and GMHA-Col (FIG. 11C) compared to GMHA-Fib (FIG. 11A) where cells are seen to spread on hydrogel surfaces. AlamarBlue metabolic results (FIG. 11D) show a significantly higher normalized absorbance of cells grown on GMHA-Fib (4989±840) compared to GMHA (3131±720) or GMHA-Col (2247±420). Further, the percent area covered (FIG. 11E) by RSCs on GMHA and GMHA-Col hydrogels was approximately 12.9±7.8% and 13.0±11.7%, respectively, compared to GMHA-Fib hydrogels with 52.2±10.9% area covered. There do not appear to be any significant differences between metabolic activity or area covered between GMHA or GMHA-Col groups.

### Discussion

This work focused on the development of a GMHA-Fibrinogen bioconjugate that can be used to enhance hydrogel functional properties from a previously utilized GMHA formulation. GMHA is an attractive hydrogel chemistry because it is modified from hyaluronan, a naturally occurring molecule found within most tissues in the body. It can readily form crosslinked networks in the presence of UV light through methacrylate-methacrylate free radical polymerization, which provides a high level of mechanical tunability (Leach, J. B., et al. Biotechnol. Bioeng. 2003 82:578-589; Spearman, B. S., et al. J. Biomed. Mater. Res. 2020 108:279-291). Despite these advantages, GMHA does not inherently possess cell adhesive amino acids and has been shown to have slow degradability when implanted in vivo (Kasper, M., et al. Biomaterials. 2021 279:121212), thus the incorporation of a protein linker is advantageous to addressing current hydrogel limitations. The co-localization of fibrinogen observed with hyaluronan in native tissue makes it a naturally suitable linker for hyaluronan-based hydrogels. The incorporation of the whole fibrinogen protein provides multiple functional properties that are desirable to tissue engineered scaffolds, including degradability and cell adhesivity.

Conjugation of fibrinogen to GMHA can be achieved through a Michael-addition reaction between thiol groups on fibrinogen cysteine sites and GMHA methacrylate groups (Dikovsky, D., et al. Biomaterials. 2006 27:1496-1506; Nair, D. P., et al. Chem. Mater. 2014 26:724-744; Rydholm, A. E., et al. Biomaterials. 2005 26:4495-4506; Rizzi, S. C. et al. Biomacromolecules. 2006 7:3019-3029). The formation of a GMHA-Fib conjugate was confirmed through dynamic light scattering analysis. Compared to its individual GMHA and fibrinogen constituents, GMHA-Fib had a much larger hydrodynamic radius and, accordingly, a smaller diffusion coefficient. These results are indicative of formation of a larger molecule. GMHA is approximately 2 MDa in size and possesses on average 20 methacrylate sites per HA molecule (Spearman, B. S., et al. J. Biomed. Mater. Res. 2020 108:279-291). Fibrinogen is approximately 340 kDa and possesses 29 cysteine sites (Zhang, J. Z., et al. J. Biol. Chem. 1996 271:30083-30088; Kattula, S., et al. Arterioscler. Thromb. Vasc. Biol. 2017 37: e13-e21). This provides ample opportunity for successful linkages to form.

Conjugation of GMHA-Fib is performed with a molar excess of GMHA, and thus an excess of methacrylate groups, available for subsequent crosslinking to form stable hydrogel scaffolds. Photocrosslinked GMHA-Fib hydrogels shown to form soft, clear hydrogels. Fibrinogen possesses a thrombin cleavable substrate that forms the classic wound healing fibrin “clot” (Kattula, S., et al. Arterioscler. Thromb. Vasc. Biol. 2017 37: e13-e21). To determine if fibrinogen still retained its fibrin crosslinking activity after chemical synthesis, GMHA-Fib hydrogels were placed in a thrombin crosslinking solution, where hydrogels exhibited a geometric shrinkage and change in opacity, an indicative sign of fibrin crosslinking. Turbidity measurements of hydrogel precursor solution and fully UV crosslinked hydrogels placed in thrombin were taken to determine the ability for fibrin to form stable networks after conjugation. Precursor solution mixed with thrombin did not exhibit any considerable changes in absorbance, whereas UV crosslinked hydrogels showed a three-fold increase in absorbance, suggesting that steric hindrance of GMHA prevents formation of fibrin unless first stabilized within an organized hydrogel matrix (LeBoeuf, R. D., et al. Biochemistry. 1987 26:6052-6057). These findings demonstrate that fibrinogen still maintains its fibrin functional activity after chemical modification.

Physical assessment of GMHA-Fib was performed at three concentrations 20, 40, 60 mg/ml (20GMHA-Fib, 40GMHA-Fib, and 60GMHA-Fib, respectively) and compared to 20 mg/ml GMHA (20GMHA), a composition previously used in past studies. SEM micrographs show morphological changes with increasing concentration of GMHA-Fib. 20GMHA-Fib shows a porous and generally non-fibrous morphology. Porous structures can be visualized in 40GMHA-Fib with some fibrous features, with the most fibrous topology and fewest pores observed for 60GMHA-Fib. Increasing fibrous morphology can be attributed to a greater fibrinogen concentration. Comparatively, 20GMHA has a smooth surface topology with few pores visualized. Swelling analyses were used to measure effective crosslinking density and mesh size. As expected, increasing polymer concentration of GMHA-Fib results in increasing crosslinking density and decreasing mesh size. These results can be corroborated with trends seen in SEM micrographs, where fewer pores are visualized in higher concentration hydrogels. Even at the highest concentration of GMHA-Fib, the effective crosslinking density is still significantly lower than the GMHA control. The high number of crosslinks observed in GMHA compared to GMHA-Fib conjugated hydrogels is likely attributed to steric hindrance introduced by incorporation of fibrinogen that prevents methacrylate-methacrylate polymerization.

Hydrogel mechanical properties were characterized using rheological measurements and bulk indentation. The combined data reported by both modalities of testing illustrate interesting mechanistic behaviors of bioconjugated hydrogels that may be useful in their application. In general, both rheology and indentation show an increase in mechanical stiffness with increasing GMHA-Fib concentration, yet significantly lower stiffness of all GMHA-Fib groups compared to GMHA only hydrogels. This information aligns with swelling data, demonstrating that the addition of the fibrinogen linker results in fewer crosslinks and a direct decrease in stiffness. Interestingly, there were no significant differences seen in the mesh size of 60GMHA-Fib hydrogels compared to 20GMHA hydrogels, yet there appear to be significant differences between mechanical properties. This could be useful in engineering a hydrogel with softer mechanical properties, but similar physical properties, for instance, to slow degradation. Rheological frequency sweeps of GMHA-Fib hydrogels reveal softer mechanical properties at lower frequencies and stiffer mechanical properties at higher frequencies. This behavior may be attributed to molecular clustering, where at slower mechanical loading the hydrogels remain in an ordered structure and at faster mechanical loading, sterically stable GMHA-Fib molecules are forced out of their equilibrium positioning and forced into compressed clusters with large repulsive forces. Moreover, GMHA and GMHA-Fib hydrogels exhibit viscoelastic behavior as determined by a decrease in the strain-independent steady-state modulus from the strain-dependent instantaneous modulus. Most biological tissues, including nerve tissue, exhibit viscoelastic behavior, thus hydrogels with such behavior are useful for host-tissue integration.

As previously mentioned, a significant challenge with GMHA hydrogels is its slow degradability observed in vivo. It was hypothesized that the incorporation of a fibrinogen linker would provide additional degradability to hyaluronan-based hydrogels. To test this hypothesis, hydrogels were placed in either a solution containing hyaluronidase or a solution containing hyaluronidase and plasmin, which are responsible for degrading hyaluronic acid and fibrinogen, respectively. Increasing the polymer concentration of GMHA-Fib hydrogels resulted in slower hydrogel clearance regardless of which solution they were placed in, which aligns with previous data obtained from physical and mechanical data. GMHA only hydrogels maintained over 95% of their weight regardless of which solution they were placed, corroborating previous observations showing slowed hydrogel clearance from tissue during in vivo nerve regeneration in rodents (Kasper, M., et al. Biomaterials. 2021 279:121212; Lacko, C. S., et al. J. Neural Eng. 2020 17). Importantly, these data show accelerated degradation of GMHA-Fib placed in the dual-enzyme solution compared to hyaluronidase alone. 40GMHA-Fib hydrogels placed in hyaluronidase maintained approximately 11% of their initial weight by day 90, whereas 40GMHA-Fib samples placed in the dual solution demonstrated full breakdown by day 30, over three times faster than hyaluronidase alone. These results support the hypothesis that the addition of a fibrinogen linker contributes to scaffold degradation via active enzymatic breakdown.

GMHA hydrogels are biocompatible, however they do not contain cell adhesive amino acids and thus do not support robust survival of cells without the inclusion of growth permissive components. Previous work has focused on the incorporation of collagen I (GMHA-Col) as an interpenetrating network to support cells within hyaluronan-based hydrogels (Singh, I., et al. J. Colloid Interface Sci. 2019 1-12; Lacko, C. S., et al. J. Neural Eng. 2020 17). Limitations of this approach include cost, time required for both UV and thermal crosslinking, and collagen elution from not being chemically bound to the matrix. Moreover, the interpenetrating network does not contribute to hydrogel degradation and instead adds an additional matrix required for breakdown (Kasper, M., et al. Biomaterials. 2021 279:121212). To assess the biocompatibility of GMHA-Fib, rat Schwann cells were grown on top of hydrogels and their viability was compared against GMHA and GMHA-Col hydrogels using live/dead and Alamarblue analyses. To eliminate potential effects of physical properties on cellular outcomes, 60GMHA-Fib was compared against 20GMHA and 20GMHA-Col hydrogels since 60GMHA-Fib and 20GMHA were shown to have more similar physical and mechanical properties than other GMHA-Fib formulations.

Metabolic activity was significantly higher in cells grown on GMHA-Fib surfaces compared to GMHA-Col or GMHA alone. Additionally, live/dead analyses show greater numbers of cells on top of GMHA-Fib samples compared to GMHA or GMHA-Col after 14 days. Schwann cell affinity towards fibrinogen may be explained by the inherent role fibrin plays in the nerve wound healing process. After nerve injury, fibrin cables are formed to provide a chemotactic and physical cue for Schwann cells to migrate and reestablish the matrix for regenerating axons (Williams, L. R., et al. J. Comp. Neurol. 1983 218:460-470). Although collagen is highly present in native nerve tissue and is deposited by Schwann cells during tissue repair, collagen is a later stage marker for regeneration compared to fibrin, and likely does not stimulate as great an effect on growth and proliferation as fibrin. These results motivate use of GMHA-Fib hydrogels in in vivo biomedical applications, specifically neural applications, as they not only support growth of cells, but promote growth compared to previous GMHA formulations.

### CONCLUSION

Natural-based biomaterials present many advantages for tissue engineering and regenerative medicine; however, they often require modifications to provide mechanical robustness and control over scaffold formation that can commonly compromise desirable biological characteristics by masking functional epitopes for cell binding or creating covalent linkages that are not inherently digestible by endogenous cells. This work focuses on the development of a novel hydrogel chemistry, GMHA-Fibrinogen, which conjugates two biomaterials to not only maintain both molecules' natural characteristics but exploit their structures for use as a tissue-engineered scaffold.

GMHA-Fib hydrogels can be easily fabricated through a Michael-addition reaction and maintain the “clotting” activity of fibrin after conjugation. In comparison to previous GMHA only formulations, GMHA-Fib exhibits significantly softer mechanical properties that are within the range of soft tissue and exhibit mechanical tunability with increasing concentration. Fibrinogen linkers within GMHA-Fib hydrogels maintain their bioactivity by not only promoting cell adhesion and growth on scaffold surfaces, but also accelerate scaffold degradation when placed in enzymatic solutions containing hyaluronidase at concentrations similar to those in nerve tissue. Future work will focus on the further development of GMHA-Fib hydrogels for other tissue engineering applications.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A hydrogel comprising hyaluronic acid polymers at least partially crosslinked with fibrinogen molecules.

2. The hydrogel of claim 1, wherein the hyaluronic acid polymer is partially crosslinked with glycidyl methacrylate.

3. The hydrogel of claim 1, wherein the hydrogel does not comprise collagen.

4. A composition comprising a glycidyl methacrylate hyaluronic acid monomer chemically conjugated to a fibrinogen molecule.

5. The composition of claim 4, wherein the fibrinogen molecule is conjugated to the glycidyl methacrylate hyaluronic acid monomer by a click-chemistry reaction.

6. The composition of claim 5, further comprising a photoinitiator.

7. The composition of claim 6, wherein the photoinitiator comprises 1-[4-(2-hydroxyethoxy)-phenyl]-2-hydroxy-2-methyl-1-propanone (Irgacure 2959) or lithium phenyl-2,4,6-trimethylbenzoylphosphinate (LAP).

8. A method for producing a hydrogel, comprising combining the composition of claim 4 with an effective amount of a photoinitiator and ultraviolet light to crosslink unconjugated methacrylate sites through free-radical polymerization.

9. The method of claim 8, wherein the photoinitiator comprises 1-[4-(2-hydroxyethoxy)-phenyl]-2-hydroxy-2-methyl-1-propanone (Irgacure 2959) or lithium phenyl-2,4,6-trimethylbenzoylphosphinate (LAP).

10. A method for treating a subject with peripheral nerve injury, comprising implanting the hydrogel of claim 1 at the site of the injury to promote neural regeneration.

11. The method of claim 10, wherein the peripheral nerve injury involves a traumatic accident, a vascular disease, or tumor excision.

12. The method of claim 11, wherein the vascular disease is a peripheral arterial disease.

13. The method of claim 12, wherein the subject has diabetes.

14. The method of claim 10, wherein the hydrogel is wrapped in a nerve guidance conduit prior to implantation.

15. The method of claim 14, wherein the nerve guidance conduit comprises decellularized porcine small intestine submucosa.
